(19) United States
(12) Patent Application Publication
(10) Pub. No.: US 2011/0288463 A1
(43) Pub. Date: Nov. 24, 2011

(54) MEDICAL PACKAGE INCLUDING ADHESIVE DRESSING

(75) Inventors: Roy John Girasa, Beaverton, OR (US); Lance David Hopman, Tigard, OR (US); Jaret Dwayne Shaffer, Lake Oswego, OR (US)

(73) Assignee: The Seaberg Company, Inc.

(21) Appl. No.: 13/037,233

(22) Filed: Feb. 28, 2011

Related U.S. Application Data
(60) Provisional application No. 61/396,106, filed on May 20, 2010.

(51) Int. Cl. A61F 13/14; B65B 5/02; A61F 15/00

(52) U.S. Cl. 602/57; 206/440; 53/452

(57) ABSTRACT

A package for protecting sterile items for medical use and including an adhesive wound dressing as a part of the package. The package may be constructed as a pair of interconnected sheets of flexible material that can be torn apart to open the package and release items contained within. A layer of adhesive material is provided on an inner face of at least one of the sheets of flexible material, protected by a release sheet that can be removed to expose the adhesive, so that the sheet of flexible material can be used as an adhesive dressing.

### BACKGROUND OF THE INVENTION

The present application is concerned with medical supplies, and in particular with packaging for medical items including wound dressings for use in caring for trauma victims.

Strong flexible, watertight packaging is used to protect sterile medical items such as bandages, various types of dressings, tourniquets, surgical gloves, splints, and other single-use items that must be available in the field for use in emergency trauma situations by medical care providers such as ambulance personnel, emergency room personnel, or military medical personnel operating in combat.

Strong protective packages are routinely used to carry special adhesive dressings for chest wounds and may also be used to carry and preserve sterility of large adhesive bandages, for emergency closure of large abdominal wounds such as those that might be suffered in combat, or for dressing burns that may be suffered in automobile accidents or in military combat.

It is desired to make better use of such packaging and to reduce the number of different special packages needed to be carried by a caregiver in order to be prepared to deal effectively with various types of potential trauma.

### SUMMARY OF THE INVENTION

In response to the need described above, the present disclosure describes inventions as defined by the claims forming a part of this disclosure. The present disclosure provides a package for medical items that can also serve as an adhesive wound dressing, and also provides a method for using a part of a package for sterile medical supplies as a wound dressing.

In one preferred embodiment a package as disclosed herein may be in the form of an envelope having a pair of sheets of strong flexible material joined to each other by an adhesive strip or heat sealed adhesively to each other around the periphery of each sheet to contain one or more sterile medical items. Such sheets may be of metal foil reinforced with a plastic such as Mylar. A layer of an adhesive material may be provided in an area on an inner face of at least one of the sheets forming the package, with the adhesive material covered protectively by a release sheet so that items carried within the package will not adhere to the adhesive, but allowing the adhesive material to be used to fasten such a sheet of the package to a patient as a protective dressing after the package has been opened.

In one embodiment the adhesive material may include a hydrogel adhesive material.

According to the method disclosed herein an item for medical trauma care, such as a sterile dressing of a special type, may be transported in a within a package, and after the package has been opened to permit use of the enclosed item a release sheet may be removed from a layer of an adhesive material on an inner face of one sheet of the package itself, and that sheet may be used as an adhesive dressing.

The foregoing and other features of the invention will be more readily understood upon consideration of the following detailed description of the invention taken in conjunction with the accompanying drawings.

### BRIEF DESCRIPTION OF THE SEVERAL DRAWINGS

FIG. 1 is an isometric view of a package for medical items which is an embodiment of the present invention.

FIG. 2 is a sectional view, taken along line 2-2 of FIG. 1 showing the construction of the package and an adhesive dressing incorporated in one of the sheets of which the package is formed.

FIG. 3 is an isometric view of the package illustrated in FIG. 1, showing the package partially opened, with items removed from within the package, and showing the included adhesive dressing.

FIG. 4 is a pictorial view showing the adhesive dressing incorporated in the package shown in FIGS. 1-3 in use on the back of a patient.

FIG. 5 is an isometric view of a package similar to that shown in FIGS. 1-3 and which incorporates a pair of adhesive dressings.

FIG. 6 is a view of a package containing a dressing adhesively attached to a release sheet which protects an adhesive layer of the dressing, as well as protecting an adhesive layer of a dressing incorporating a sheet of the package itself

FIG. 7 is an isometric view showing an IV kit contained in a package such as that shown in FIG. 1.

FIG. 8 is an isometric view showing a package such as that shown in FIG. 1 in use to contain a large dressing.

FIG. 9 is a view similar to FIG. 8 showing a package such as that shown in FIG. 1 used to contain a splint.

### DETAILED DESCRIPTION OF PREFERRED EMBODIMENTS

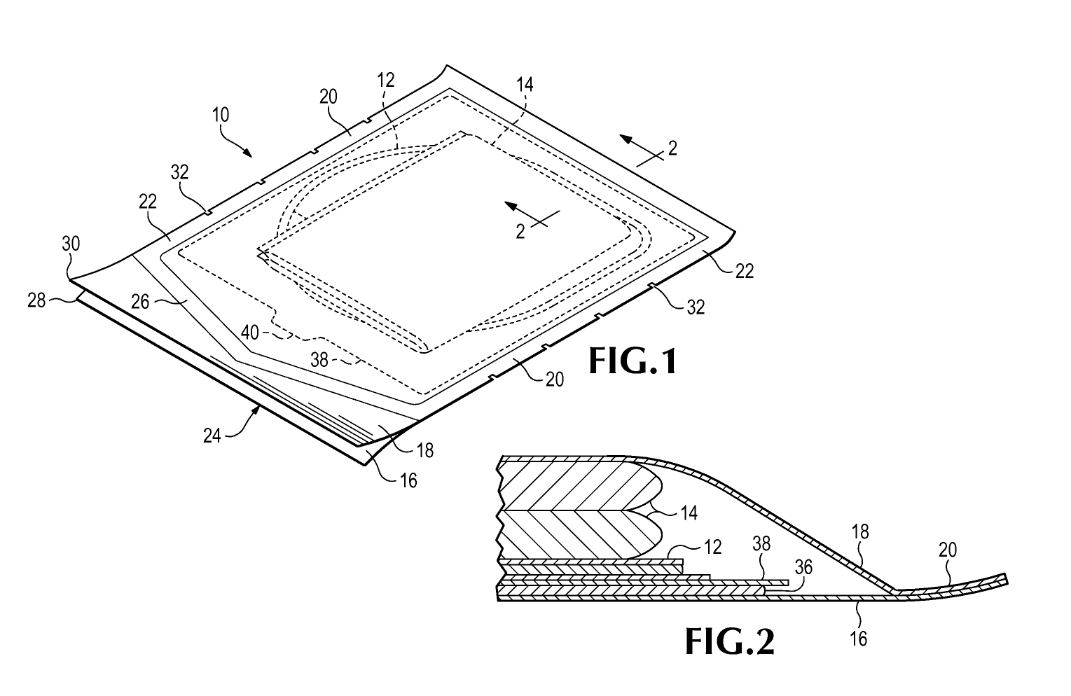

Referring now to the drawings which form a part of the disclosure herein, a package 10 shown in FIG. 1 encloses and protects in a sterile condition a chest seal dressing 12 and an absorbent pad 14 of folded gauze material.

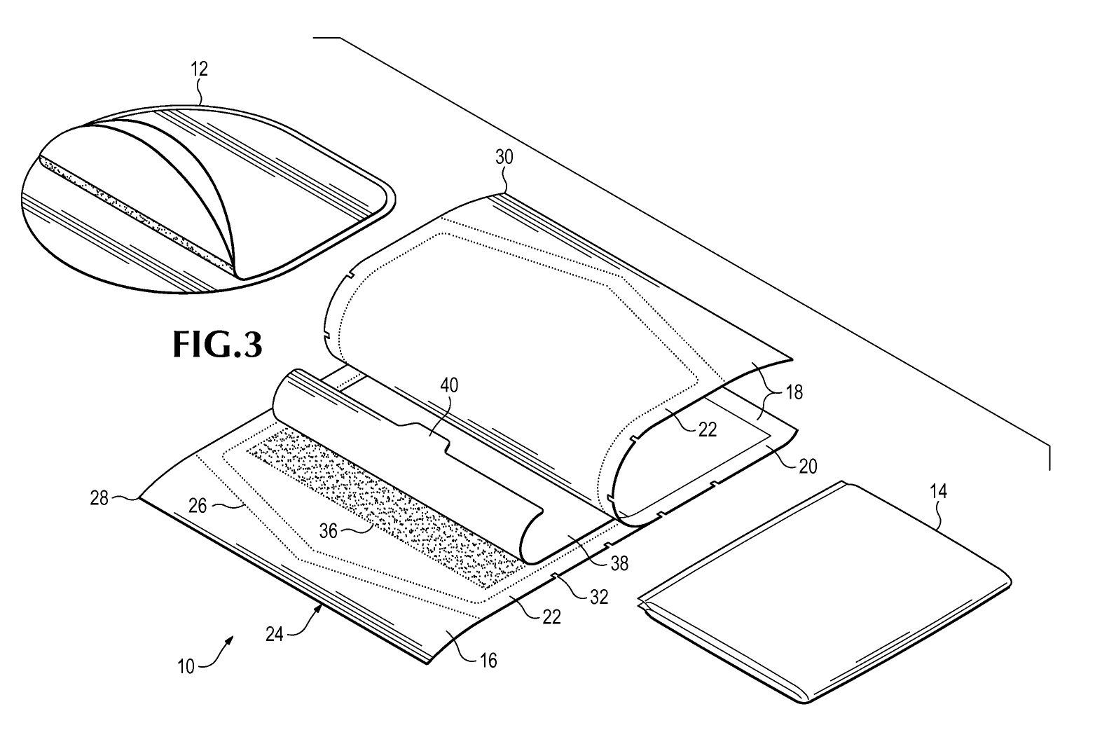

Referring also to FIGS. 2 and 3, the package 10 is seen to include a first, bottom, or base, sheet 16 which may be of metal foil reinforced with a plastic such as Mylar. The base sheet 16 may be also be of a suitably strong paper impregnated or coated with a plastic or other suitable material to make the sheet gastight and watertight, or the base sheet 16 may be entirely of a suitable plastics material. A second, upper, or front sheet 18 may be of similar foil laminate, paper-and-plastic laminate, or plastic material, and in some cases it may be desirable for the front sheet 18 to be transparent.

As shown in section view in FIG. 2 the various layers of the package 10 and its contents are exaggerated in thickness for clarity. The package 10 is preferably sealed shut with a minimum amount of air or other gas included, to avoid bulging in hot environments, instead of having as much room inside as is shown in FIG. 2. As may be seen best in FIG. 3, the front sheet 18 is connected to the base sheet 16 in a connection zone 20, which may be in the form of a connecting band 22 of an adhesive material interconnecting the front sheet 18 and the base sheet 16 along the peripheral margins of the two sheets 16 and 18 defining a receptacle between the sheets 16 and 18 and within the protected area defined by the connection zone. The connecting band 22 of adhesive material may conveniently be spaced apart from an edge of the sheets 16 and 18 of flexible material at a first, or top end 24 of the package 10, for example as a chevron-shaped portion 26 of the band 22, allowing the corners 28, 30 of the sheets 16, 18 to be grasped easily in order to pull the front sheet 18 apart from the base sheet 16 to open the package 10 by overcoming the connection of each sheet to the other in the connection zone 20, as by breaking the adhesive connection.

The connection of the front sheet 18 to the base sheet 16 in the connection zone 20 may be accomplished using well-known chemical adhesives or by heat-sealing, that is, thermal welding, by well known means along the connecting band 22. In either case, the connection of the two sheets to each other in the connection zone 20 is preferably strong enough to provide an adequate seal to protect the sterility of items contained within the package 10, but weaker than either of the sheets 16 and 18, so that the sheets can be separated from each other without destroying either sheet.

As an alternative way to open the package 10, tear notches 32 may be provided along one or more of the margins of the package 10.

In the package 10 as shown in FIGS. 1-3, the chest seal dressing 12 may be of a type designed for closing wounds such as those which might be suffered by gunshot victims or that may be inflicted by grenade fragments or other shrapnel in a combat situation. In some cases a bullet may enter a person's body from the front and leave the rear of the person's body, causing an exit wound. The chest seal dressing 12 may be of a type specifically designed as a chest seal for a sucking chest wound (tension pneumothorax) and may in some cases include a check valve to be used to prevent collapse of a patient's lung. While it is of primary importance to close and seal such a primary chest wound, it is also important to close an exit wound and provide some protection for the area surrounding the exit wound in a situation where a projectile has passed entirely through a person's body. In order to avoid having to open a second package and utilize a bandage contained in such a second package to deal with an exit wound in such a patient the package 10 includes a layer 36 of adhesive material located on a protected area of the inner face of the base sheet 16, surrounded by the connection zone 20 and thus protected in a sterile condition within the package 10 so long as the package remains unopened. The adhesive layer 36 thus may be nearly as large as the entire area surrounded by the connecting band 22 in which a thermal weld or adhesive material is used to join the sheets 16 and 18 to each other. The layer 36 may be of one of various types of medical adhesives such as rubber-based adhesives, acrylic-based adhesives, hydrocolloid adhesives, and hydrogel adhesives.

A release sheet 38 is applied to the layer 36 of adhesive material and should be of a great enough size to extend a small distance beyond the edge of the layer 36 of adhesive material on all sides. A grip tab 40 may be provided on the release sheet extending a greater distance beyond the edge of the adhesive material 36 as a convenient way to grasp the release sheet 40 to remove it without contaminating the surface of the layer 36 of adhesive material.

The package 10 may be opened by a medical caregiver by grasping the corners 28 and 30 of the sheets 16 and 18, pulling them apart to strip the front sheet 18 away from the base sheet 16, exposing medical items within the package 10, such as the chest seal dressing 12 and the gauze pad 14. With the front sheet 18 and packaged item removed from the first or base sheet 16, the release sheet 30 can be peeled away from the layer of adhesive 36, so that the base sheet 16 is available as a large adhesive dressing that is airtight and watertight and can be attached by pressing the adhesive material onto a patient's body where there may be an exit wound or multiple wounds, or where skin has been lost and tissue needs to be protected from the conditions of the environment.

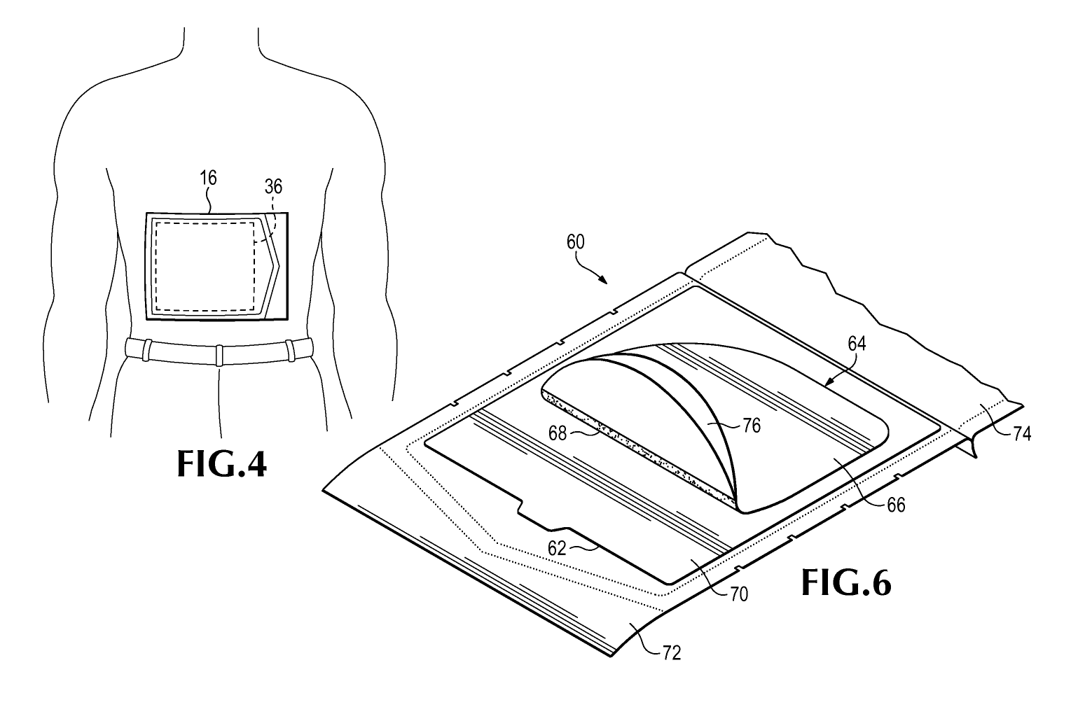

The package 10 may be particularly appropriate for gunshot wound victims, military personnel injured in combat, automobile accident victims, and others, containing different types of bandages which may commonly be needed for a victim of such trauma incidents. The base sheet 16 is also available to be applied to a patient's body, as to cover an exit wound, as shown in FIG. 4, using the layer of adhesive material 36 to attach the base sheet 16 to the person's body.

As shown in FIG. 5, in a package 46 otherwise similar to the package 10, both a first, base sheet 48 and a second, front sheet 50 may each be provided on its inner face with a respective layer of adhesive 36 protected by a respective release sheet 38. When the package 46 is opened any medical items stored within the package 46 are made available, and two additional impermeable adhesive dressings are also available in the form of the two sheets 48 and 50 forming the package 46.

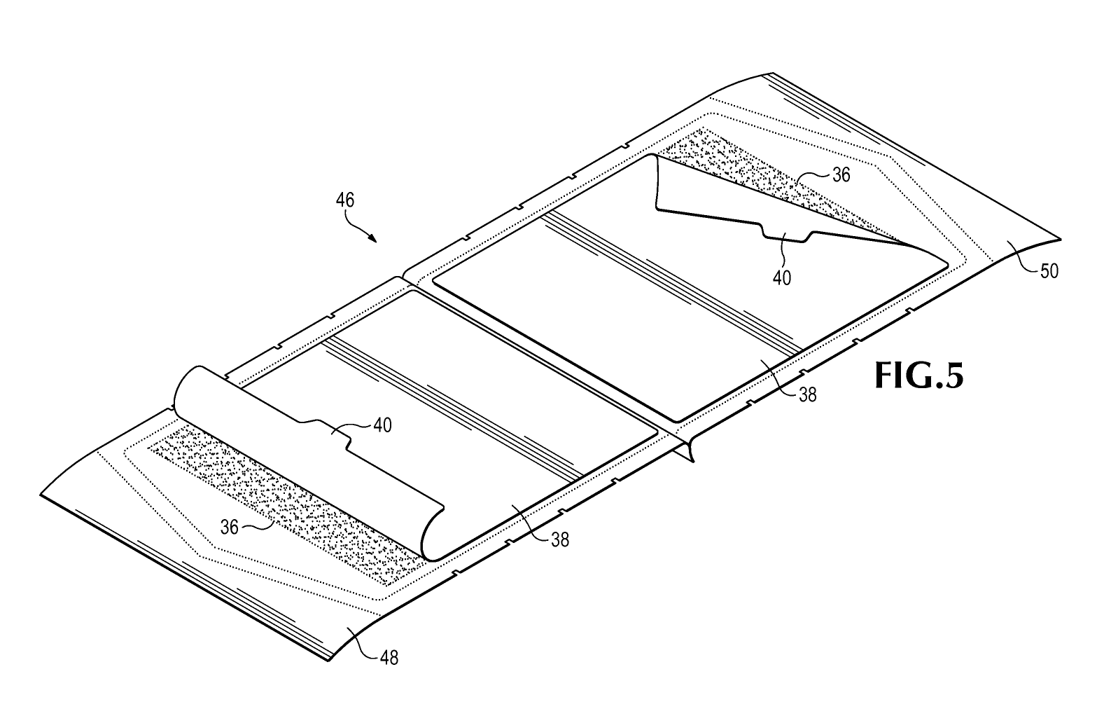

As shown in FIG. 6, in a package 60 which is another variation of the package 10, a release sheet 62 is of a type having a releasable surface on each of its two opposite faces. An adhesive dressing such as a chest seal dressing 64, having a flexible layer of sheet plastic 66 carrying a layer of a suitable medical adhesive 68 to attach the chest seal 64 to a patient's skin, is attached to the outer face 70 of the release sheet 62 by the adhesive 68. The chest seal 64 can be peeled away from the release sheet 62 as shown in FIG. 6 for use of the chest seal dressing 64 once the package 60 has been opened. A folded piece 76 of release paper is carried on a part of the chest seal 64 for use first in peeling it away from the package 60 and for later use in placing the chest seal where needed on a patient. Thereafter, the release sheet 62 may be pulled away from the layer 36 of adhesive carried on the first or base sheet 72, so that a first or base sheet 72 of the package 60 may be used as an adhesive dressing in the same manner described above with respect to the use of the base sheet 16 of the package 10. A front, or second sheet 74 of the package 60 may also incorporate a layer 36 of adhesive covered by a release sheet 62 and carrying another adhesive dressing attached to that release sheet.

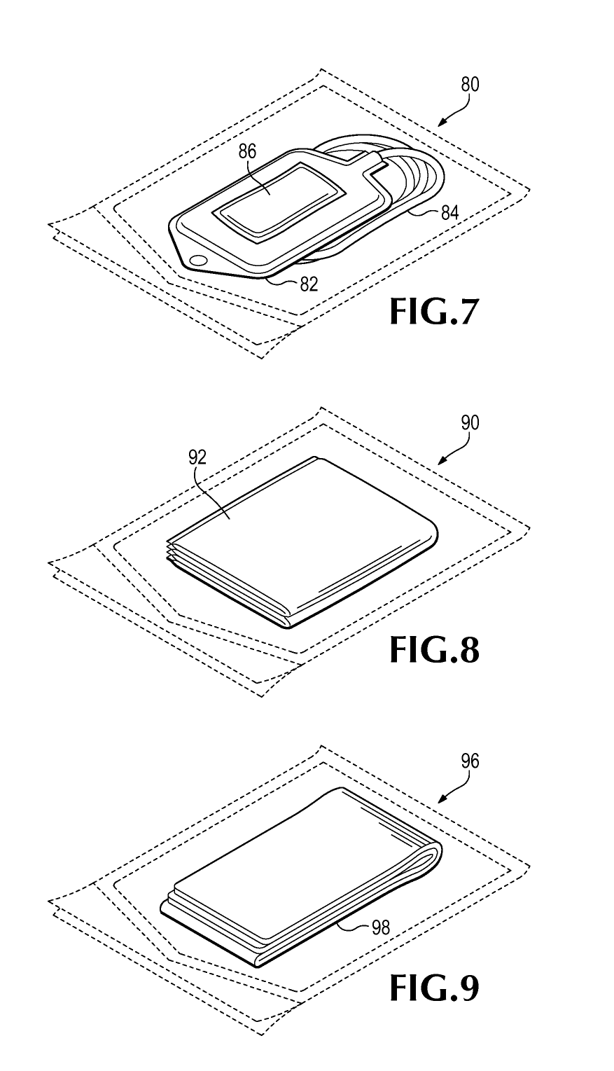

Referring now to FIGS. 7, 8, and 9, a package similar to one of the packages 10, 46, or 60 may be used effectively to contain and protect in a sterile condition various items potentially necessary in a medical emergency. For example, a package 80 may be used to contain an intravenous injection kit including a fluid bag 82, a tube 84, and package 86 of needles and clamps, etc. One or both of the sheets of flexible material forming such a package may include a layer of adhesive and a release sheet as described above so that that sheet of the package 80 may be used as an adhesive dressing in an appropriate situation.

As another example, a package 90, shown in FIG. 8, may be used to contain and protect in a sterile condition a large bandage, such as a special burn dressing 92 shown folded. One or each of the sheets forming the package 90 may include a layer of adhesive material on its inner face and an associated release sheet, so that one or both of the sheets of the package 90 may be used as adhesive dressings as explained above.

As yet another example, a package 96 shown in FIG. 9 may include a folded emergency splint 98 that can be bent to a required shape for use, while one or both of the sheets forming the package may also be used as adhesive dressings in the manner described above.

It will be understood that various versions of a package such as the package 10 may be used to contain various other items which must be kept sterile until their use is required in a medical emergency situation. For example, items such as tourniquets, cricothyrotomy kits, surgical gloves, and respirator masks might also be contained in such packages, ready for use in situations which might well also require the use of an adhesive, impermeable, dressing at least temporarily.

The terms and expressions which have been employed in the foregoing specification are used therein as terms of description and not of limitation, and there is no intention in the use of such terms and expressions of excluding equivalents of the features shown and described or portions thereof, it being recognized that the scope of the invention is defined and limited only by the claims which follow.

What is claimed is:

1. A package comprising:
(a) a first sheet of flexible material having an inner face;
(b) a second sheet of flexible material having an inner face;
(c) a connection zone through which the inner face of the second sheet is connected to the inner face of the first sheet along a respective connecting band defined on one of the sheets, the connection zone surrounding a protected area of the inner face of the first sheet and defining a receptacle between the first and second sheets of flexible material;
(d) a layer of an adhesive material attached to the inner face of the first sheet within the protected area; and
(e) a release sheet overlying and adhering to the layer of adhesive material.

2. The package of claim 1 further including at least one sterile item for medical use contained within the receptacle and isolated from the layer of adhesive material by the release sheet.

3. The package of claim 1 wherein the receptacle is sealed by the connection zone.

4. The package of claim 1 wherein the inner face of said second sheet of flexible material includes a second protected area surrounded by the respective connecting band, and wherein a second layer of an adhesive material is attached to the inner face of the second sheet within the second protected area, and wherein a second release sheet is adhered to the second layer of adhesive material.

5. The package of claim 1 further including at least one sterile item for medical use contained within the receptacle and isolated from the layer of adhesive material by the release sheet, with an adhesive layer of said sterile item adhered to a side of the release sheet facing away from the layer of adhesive material attached to the inner face of the first sheet.

6. The package of claim 1 wherein the connecting band includes a chemical adhesive.

7. The package of claim 1 wherein the connecting band includes a thermal weld interconnecting respective parts of the first and second sheets.

8. A method of packaging an item for medical use, comprising:
(a) providing a first sheet of flexible material having an inner face;
(b) providing a second sheet of flexible material having an inner face;
(c) applying a layer of an adhesive material to the inner face of the first sheet;
(d) mounting a release sheet on the layer of adhesive material;
(e) placing an article to be packaged between said release sheet and said inner face of said second sheet; and
(f) connecting said inner face of said first sheet to said inner face of said second sheet along a continuous connecting band encircling said layer of adhesive and said release sheet and thereby forming a receptacle between said first and second sheets and enclosing said article within in said receptacle.

9. The method of claim 8 wherein said step of connecting includes forming a thermal weld interconnecting respective parts of said first and second sheets.

10. A method of providing an adhesive covering, comprising:
(a) obtaining a package including:
(i) a first sheet of flexible material having an inner face,
(ii) a second sheet of flexible material having an inner face,
(iii) a connection zone through which the inner face of the second sheet is connected to the inner face of the first sheet surrounding a protected area of the inner face of the first sheet and defining a storage receptacle between the first and second sheets of flexible material,
(iv) a layer of an adhesive material attached to the inner face of the first sheet within the protected area, and
(v) a release sheet overlying and adhering to the layer of adhesive material; and performing the further steps of:
(b) pulling said second sheet free from said first sheet;
(c) removing said release sheet from said layer of adhesive material;
(d) placing said first sheet of flexible material in a desired location adjacent a surface to be protected; and
(e) fastening said first sheet of flexible material protectively in place on the surface to be protected by placing said adhesive material into contact against said surface.

11. The method of claim 10 including the step of removing an article from within the package prior to said step of removing said release sheet from said layer of adhesive material.

12. The method of claim 11 wherein removing an article from within the package includes peeling the article from a release sheet adhering to the layer of adhesive material attached to the inner face of the first sheet of flexible material.

13. The method of claim 10 wherein said step of pulling said second sheet free from said first sheet of flexible material includes tearing said second sheet of flexible material apart from said first sheet of flexible material along said connection zone.
